(19) United States
(12) Patent Application Publication
(10) Pub. No.: US 2014/0018782 A1
(43) Pub. Date: Jan. 16, 2014

(54) System, Method, and Apparatus for Performing Surgery using High Power Light Energy

(71) Applicant: Gregory Flynn, Tampa, FL (US)

(72) Inventor: Gregory Flynn, Tampa, FL (US)

(21) Appl. No.: 14/031,476

(22) Filed: Sep. 19, 2013

Related U.S. Application Data
(63) Continuation-in-part of application No. 13/074,454, filed on Mar. 29, 2011, now abandoned.

(51) Int. Cl. A61B 18/22

(52) U.S. Cl. CPC A61B 18/22
USPC 606/3

(57) ABSTRACT

A system, method, and apparatus for performing surgery within the epidural space of a patient includes at least two high-power light generators. The epidural space is filled with a fluid to maintain patency and to absorb excess light power. The light energy from the high-power light generators is directed into a first end of a bundle of fiber optics and the second end of the bundle is maneuvered within the epidural space of the patient. When the second end of the fiber optics is aimed at unwanted tissue of a first type, the appropriate high-power light generator is operated to vaporize that tissue. When the second end of the fiber optics is aimed at unwanted tissue of a second type, the appropriate high-power light generator is operated to vaporize that tissue.

### CROSS-REFERENCE TO RELATED APPLICATION

This application is a continuation-in-part of U.S. patent application Ser. No. 13/074,454, filed Mar. 29, 2011, the disclosure of which is hereby incorporated by reference.

### FIELD

This invention relates to the field of medicine/surgical devices and methods and more particularly to a system/device and method for performing surgery with the use of high-power light.

### BACKGROUND

Less invasive or “minimally invasive” surgical techniques have become increasingly popular, as physicians, patients and medical device innovators seek to reduce the trauma, recovery time and side effects typically associated with conventional surgery. The art of such less invasive surgical methods and devices has many challenges. For example, less invasive techniques involve working in a smaller operating field, working with smaller devices, and trying to operate with reduced or even no direct visualization of the structures being treated. These challenges are often compounded when target tissues of a given procedure reside very close to one or more vital, non-target tissues.

Many areas of surgery have moved from the traditional operating procedures to less invasive procedures. For example, in many cases, a gallbladder is removed through a tiny incision.

One area of surgery that has benefited from less invasive techniques is the treatment of spinal stenosis. Spinal stenosis occurs when nerve tissue and/or the blood vessels supplying nerve tissue in the spine become infringed upon by one or more structures in the lower spine leading to pain, numbness and/or loss of certain functions.

In the United States, spinal stenosis is frequent in adults aged 50 and older and is the most frequent reason cited for back surgery in patients aged 60 and older. Often, due to their weight and unsymmetrical weight characteristics, obese people are more apt to suffer from spinal stenosis.

Patients suffering from spinal stenosis are often treated with exercise therapy, analgesics, anti-inflammatory medications, and epidural steroid injections. When these conservative treatments do not work or the patient's symptoms are severe, surgery may be required to remove the infringing tissue and decompress the impinged nerve tissue.

Lasers have proven themselves incredibly valuable in lumbar spinal stenosis surgery. Prior to the use of lasers, an incision was made in the back, and muscles and supporting structures were stripped away from the spine to expose the vertebral column. Complete or partial removal of any bony arch covering the back of the spinal canal may then be performed. In addition, the surgery often includes partial or complete removal of all or part of one or more facet joints to remove infringing ligamentum flavum or bone tissue. Such spinal stenosis surgery was performed under general anesthesia and the patients required a five to seven day hospital stay, with full recovery taking between several weeks to three months. Therapy at a rehabilitation facility was often required to regain desired mobility.

Less invasive surgical methods and devices for treating spinal stenosis and other back problems often utilize a laser to remove the infringing tissue. “Epiduroscopy” by G. Schültze describes methods of performing spinal endoscopy using lasers. In this, G. Schültze describes methods for entering the epidural space, guiding a fiber optic probe into the epidural space with the help of a C-arm device and correcting various situations using the laser. In chapter 7.5, G. Schültze discusses the Epidural laser adhesiolysis, for example, using a 1064-nm Nd, YAG 1320-nm nd and a 940-nm laser for “coagulation of bleeding, rechanneling stenosis caused by tumors and destroying plaques in vessel walls.” In this, a fiber optic is introduced into the epidural space via a working channel of an epiduroscope under epiduroscope vision. A laser diode of from 1 watt to 25 watts fires a burst of energy through the fiber and onto the target tissue. G. Schültze describes that the light energy penetrates the tissues but is not significantly absorbed by the surrounding hemoglobin, melanin or water.

It is well known that different light frequencies are absorbed differently by different target materials. The described procedure uses lasers with a wavelength of from around 940-nm to 1320-nm. These wavelengths are selected because they are well absorbed by both hemoglobin and water, which are both major components of cartilage and scar tissue.

In another example of the prior art, a 532-nm (Green-light) laser has proven successful in treatment of the prostate and other urological conditions. A method referred to as Photo-Selective Vaporization has been successfully used on Benign Prostatic Hyperplasia (BPH) to remove enlarged prostate tissue, resulting in an open channel for urine flow. This specific wavelength of laser energy is selected because it is maximally absorbed by hemoglobin and, therefore, absorbed by tissue that has blood in it such as prostate tissue.

U.S. Pat. Pub. 2008/0267814 to Bornstein shows the value of multiple wavelength lasers for use in elimination of microbes. In this application, two wavelengths can include emission in two ranges approximating 850 nm to 900 nm and 905 nm to 945 nm at the same time. This application does not alternate the use the lasers depending upon the type of target tissue and not in the epidural space or spinal canal.

U.S. Pat. Pub. 2008/0103504 to Schmitz, et al, describes a method of removing ligamentum flavum tissue in the spine to treat spinal stenosis. There is no disclosure of the wavelength of laser or having multiple laser wavelengths.

U.S. Pat. Pub. 2008/0039828 to Jimenez, et al, describes using a laser of a particular wavelength specifically tuned to a biocompatible colorant. The target tissue is colored by the colorant and the laser used to vaporize the tissue that has been colored by the colorant. This disclosure describes a single laser of a wavelength that is absorbed by the colorant and, therefore, the target tissue is changed (in color) to better absorb the light energy of the fixed-wavelength laser.

What is needed is a system, method and apparatus that will selectively provide the correct wavelength of energy to the target tissue, without removal of the fiber and insertion of a different fiber, within an aqueous environment.

### SUMMARY

A system for performing laser surgery within the epidural space of a patient includes at least two high-power light generators (e.g. 1 to 25 watts each for example). The light energy from the high-power light generators is directed into a first end of bundle containing one or more fiber optics and the second end of the bundle is maneuvered within the epidural space of the patient which is filled with a fluid. When the second end of the bundle is aimed at unwanted tissue of a first type, the appropriate high-power light generator is operated to vaporize that tissue. When the second end of the bundle is aimed at unwanted tissue of a second type, the appropriate high-power light generator is operated to vaporize that tissue.

In one embodiment, a system for performing surgery within the epidural space of a patient is disclosed. The epidural space filled with a fluid before performing the surgery. The system includes a plurality of high-power light emitting devices. Each of the high-power light emitting devices emits light energy of a different wavelength of approximately 980 nanometers, 1480 nanometers and 2100 nanometers. A device controls the high-power light emitting devices, initiating output of the light energy under user control. One or more fiber optics delivers the light energy into the epidural space. A first end of the fiber optics accept the light energy from the plurality of high-power light emitting devices and a distal second end delivers the light energy to target tissue within the epidural space of the patient. A first type of tissue is vaporized using the light energy from a first high power light emitting device of the plurality of high-power light emitting devices and a second type of tissue is vaporized using the light energy from a second high power light emitting device of the plurality of high-power light emitting devices without the need to remove the fiber optics from the epidural space, thereby maintaining the fluid within the epidural space.

In another embodiment, a method of performing surgery within the epidural space of a patient is disclosed including opening a port to access the epidural space and filling the epidural space with a fluid. A high-power light source is provided. The high-power light source has a high-power light emitting devices, each of which emit light energy of a different wavelength than the other high-power light emitting devices, selected from 980 nanometers, 1480 nanometers and 2100 nanometers. Each of several switches controls a different one of the high-power light emitting devices, initiating output of the light energy by user control. A bundle of one or more fiber optics has a first end for accepting light energy from the high-power light emitting devices and a distal second end for delivering the light energy into the epidural space. The method continues with inserting the distal second end of the bundle into the epidural space of the patient and aiming the distal second end of the one or more fiber optics at a first type of tissue. A first switch of the switches is activated to emit light from a first of the high-power light emitting devices to affect the first type of tissue. Without removing of the bundle (thereby maintaining patency) the distal second end of the one or more fiber optics is aimed at a second type of tissue and a second switch of the switches is activated to emit light from a second of the high-power light emitting devices to affect the second type of tissue. The fluid within the epidural space attenuates excess energy from the plurality of high-powered light emitting devices.

In another embodiment, a system for performing surgery within the epidural space of a patient is disclosed including a device for opening an access to the epidural space and a device for substantially filling the epidural space with a fluid in order to maintain patency and to attenuate excess light energy. A first source of laser light energy emits light at 980 nanometers and has a first switch for initiating light output from the first source of laser light energy when operated. A second source of laser light energy emits light at 1480 nanometers and has a second switch for initiating light output from the second source of laser light energy when operated. A bundle of one or more fiber optics has a first end for accepting light energy from the first source of laser light energy and from the second source of laser light energy, and has a distal second end for delivering the light energy to target tissue within the epidural space of the patient. A first type of tissue is vaporized using the light energy from the first source of laser light energy and a second type of tissue is vaporized using the light energy from the second source of laser light energy without the need to remove the fiber optics from the epidural space, thereby maintaining the fluid within the epidural space.

In another embodiment, a system for performing laser surgery within the epidural space of a patient is disclosed including single or multiple high-power light generators (e.g. 1 to 25 watts each for example). Each light generator is a laser or IPL emitting CW or pulsed energy of the desired wavelength. The light energy from the high-power light generator(s) is directed into the proximal end of one or more fiber optics and the distal end of the fiber optic(s) are maneuvered within the epidural space of the patient. When the distal end of the fiber optic(s) is aimed at unwanted tissue of a first specific type, the high-power light generator(s) are operated to vaporize that specific tissue. When the distal end of the fiber optic(s) is aimed at unwanted tissue of a second specific type, the high-power light generator(s) are operated to vaporize that specific tissue. The different light wavelengths of light are emitted through a single fiber optic simultaneously, or alternatively, at variable wattages to vaporize the material causing the spinal stenosis, nerve impingement, or spinal canal obstruction.

### BRIEF DESCRIPTION OF THE DRAWINGS

The invention can be best understood by those having ordinary skill in the art by reference to the following detailed description when considered in conjunction with the accompanying drawings in which:

FIG. 1 is a perspective view of the surgery system incorporating the cushion support system and cannulated sacral introducer rasp.

FIGS. 2A and 2B illustrate the cushion support system.

FIGS. 3A through 3D illustrate the placement of individual cushions to create the cushion support system.

FIG. 4A through 4C illustrate flexible placement of the cushions that make up the cushion support system.

FIG. 5 illustrates a bottom view of the leg isolation and tool support cushion.

FIG. 6 illustrates a top view of the pelvic cushion.

FIG. 7 illustrates a prior art rasp.

FIG. 8 illustrates a side view of the cannulated sacral introducer rasp.

FIGS. 9A and 9B illustrate the cannulated sacral introducer rasp in use.

FIG. 10 illustrates a prior art surgical laser.

FIG. 11 illustrates a pair of prior art surgical lasers.

FIG. 12 illustrates the multiple wavelength surgical laser system.

FIG. 13 illustrates a block diagram of a prior art surgical laser.

FIG. 14 illustrates a block diagram for the multiple wavelength surgical laser system.

FIG. 15 illustrates a graph of absorption by materials of ranges of wavelengths of light.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures. The examples below do not purport to represent all potential examples or embodiments of the invention, with many other potential examples possible by one skilled in the arts.

As described above, less invasive surgical methods and devices for treating spinal stenosis and other back problems often utilize a laser to remove the infringing tissue. “Epiduroscopy” by G. Schültze describes such methods of performing spinal endoscopy using lasers. In this, G. Schültze describes methods for entering the epidural space, guiding a fiber optic probe into the epidural space with the help of a C-arm device and correcting various situations using a laser. In chapter 7.5, G. Schültze discusses the Epidural laser adhesiolysis, for example, using a 1064-nm Nd, YAG 1320-nm nd and a 940-nm laser for “coagulation of bleeding, rechanneling stenosis caused by tumors and destroying plaques in vessel walls.” In this, a fiber optic is introduced into the epidural space via a working channel of an epiduroscope under epiduroscope vision. A laser diode of from 1 watt to 25 watts fires a burst of energy through the fiber and onto the target tissue. G. Schültze describes that the light energy penetrates the tissues but is not significantly absorbed by the surrounding hemoglobin, melanin or water.

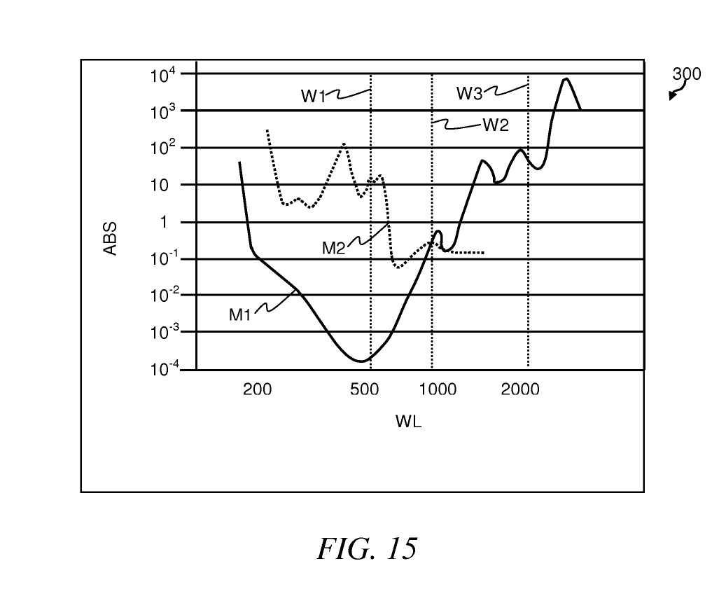

It is well known that different laser light frequencies are absorbed differently by different target materials. The described procedure in G. Schültze uses lasers with a wavelength of from around 940-nm to 1320-nm. These wavelengths are selected because they are well absorbed by both hemoglobin and water, which are both major components of cartilage and scar tissue. As shown in FIG. 15, light is well absorbed by hemoglobin up to approximately 1400 nm, while absorption by water becomes significant after 800 nm. From the point at which hemoglobin and water intersect on the absorption chart, approximately 980 nm, absorption by water increases significantly while the absorption by hemoglobin slowly decreases and becomes non-existent after 1400 nm. For this reason, different wavelengths of light energy are needed to remove tissue containing the target chromophore, or portion of the molecule responsible for the molecule's color. For example, a 532-nm (Green-light) has proven successful in vaporizing tissue containing large amounts of hemoglobin, such as enlarged prostate tissue, etc.

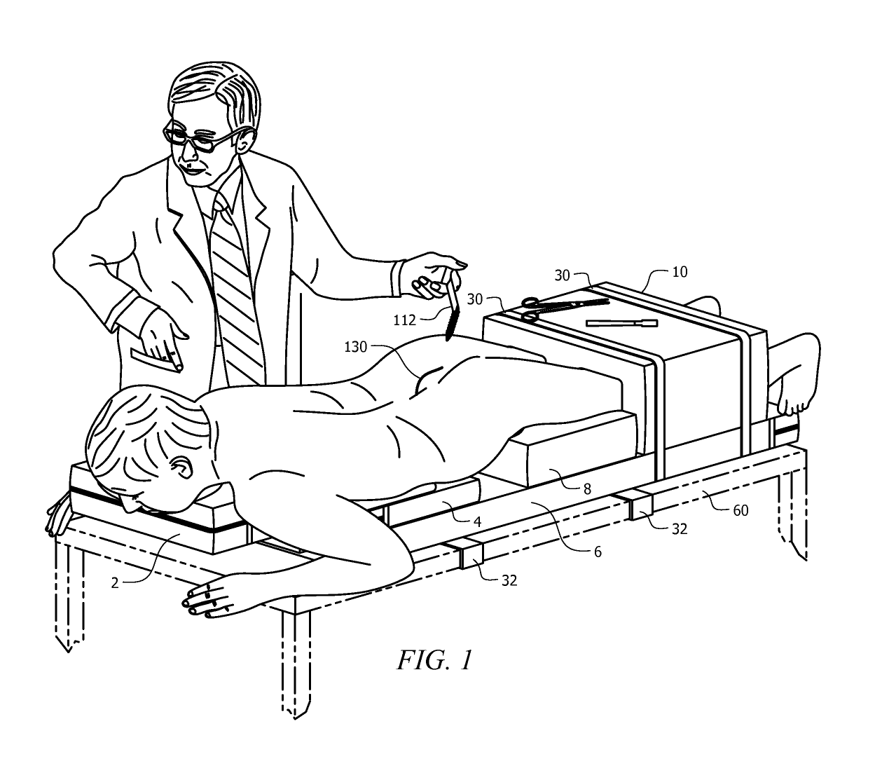

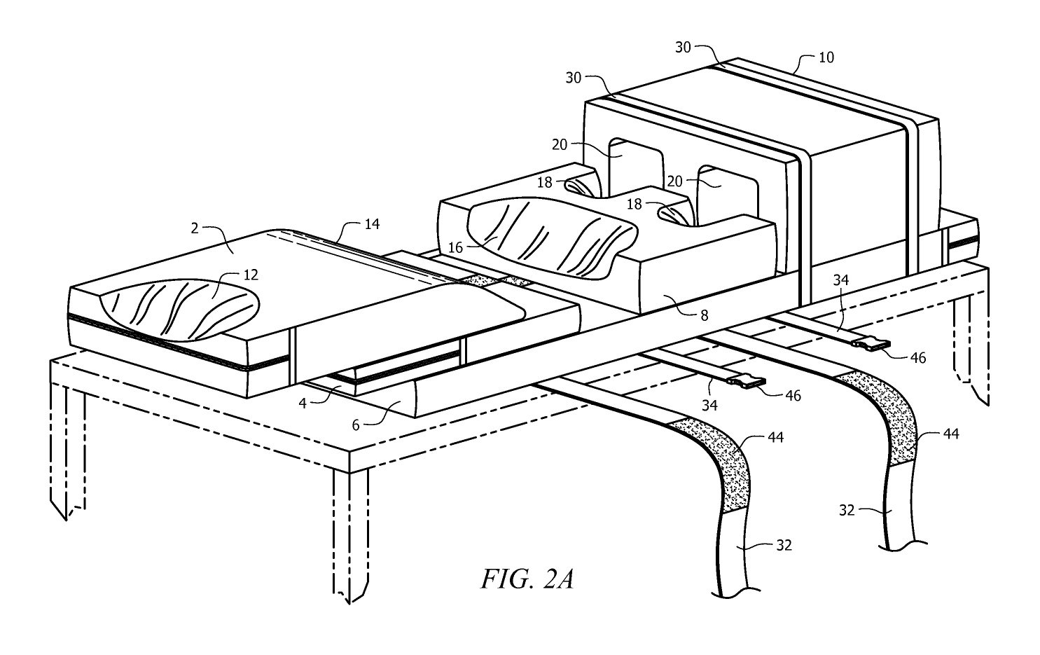

Referring to FIGS. 1, 2A, and 2B an exemplary cushion support system and cannulated sacral introducer rasp will be described. The cushion system comprises the chest support cushion 2, chest height adjustment cushion 4, main support cushion 6, pelvic cushion 8, and leg isolation and tool support cushion 10. The chest support cushion 2 includes a depression 12 for the patient's chin, and a slope 14 to support the angle of the chest. The shape of slope 14 varies for different embodiments to address the different shape of a man's chest as compared to a woman's chest. The depression 12 allows for airway access by an anesthesiologist. The cushion system comfortably positions the patient for spinal surgery. The chest support cushion 2 and chest height adjustment cushion 4 hold the chest and head, while the pelvic cushion 8 supports the pelvis, allowing the belly to hang. It is well known in the industry that many patients that have back issues are also obese. When a patient lies on their stomach, especially an obese patient, displacement of the abdomen creates higher fluid pressure in the spinal canal, particularly the fluid pressure in the epidural venous plexus surrounding the spinal nerves. Suspension of the abdomen by the disclosed cushion support system decreases this pressure, reduces the risk of side effects of surgery and provides improved access to the guide wire 130. The chest height adjustment cushion 4 is optional and is used to adjust the system for the size and shape of the patient. Whether or not the chest height adjustment cushion 4 is used, the top of the chest support cushion 2 is preferably higher than the pelvic cushion 8. This difference in height provides for having the chest and head lifted, allowing the abdomen and belly to hang, decreasing the venous pressure in the epidural veins in the spinal canal, as well as reducing the risk of retinal damage from pressure caused by use of the epiduroscope and fluid pressure infused to maintain the patency of the surgical field. The disclosed method and apparatus includes filling the epidural space with a fluid such as saline for various reasons including, but not limited to, maintaining patency of the epidural space, reducing thermal build-up, and absorbing excess laser energy. The fluid helps to float the spinal sac and fat tissue away from the camera fiber optic distal lens and increase the visibility of the fiber optic scope during use of the high power light or laser. The fluid also absorbs laser energy, greatly reducing the power of the laser energy after the light passes through approximately 0.3 mm of the fluid.

The cushion support system enables the abdominal viscera to hang freely, which creates a gravity dependent pooling of blood in the abdominal visceral blood vessels, with the most pronounced effects present in the venous system. The cushion support system creates negative pressure in the epidural veins and epidural capillaries via the connecting blood vessels. As a result, during the epiduroscopic surgical procedure the vessels are not engorged, and the risk of injury to these vessels is reduced, as well as the incidence of bleeding which obstructs the view the operative field. These benefits lower the risk of complications during back surgery.

It is anticipated that in other embodiments the effect of using the chest cushion is achieved by sloping the operating table 60 to place the level of the head above that of the pelvis. The pelvic cushion 8 has an abdominal depression 16 for a patient's belly and male genitalia, and two leg depressions 18, one for each of the patient's thighs. The leg isolation and tool support cushion 10 has two passageways 20, one for each of the patient's legs. The flat, table-top portion of the cushion 10 provides the physician a stable location for instruments. The main support cushion 6 has a plurality of straps 30/32, including straps 30 for holding the leg isolation and tool support cushion 10 in place, straps 32 for wrapping around either the patient or the operating table, and straps 34 for wrapping around the operating table. The straps removably connect in a multitude of ways such as by hook-and-loop fasteners 44, and/or buckles/snaps 46. The straps 34 keep the pad 6 removably affixed to the operating table 60.

The cushion system is best used with the head of the operating table elevated 30 degrees. This slope reduces fluid pressure in the spinal canal and reduces fluid pressure of the Cerebral Spinal Fluid (CSF), and further reduces the risk of retinal detachment by CSF fluid elevation.

In some examples, the cushion system is radiolucent, or substantially transparent to the passage of X-rays. In other embodiments the cushion system is substantially transparent to other types of signals, including those used in magnetic resonance imaging and ultrasonic imaging.

The cushions are constructed of any of a multitude of suitable materials as known in the industry. The inside of the cushion is preferably made from a supportive material such as closed-cell foam. Other inner materials are anticipated, including, but not limited to, open-cell foam, closed-cell foam, cushions of multiple material types (e.g., a stiff inner core and soft outer layer), natural and synthetic fillers, and all others as commonly known in the art. The outer covering of the cushion is preferable made from a water-resistant or water-proof fabric to facilitate cleaning. Other outer coverings are anticipated, including synthetic and natural fabrics, genuine and faux leather, and all others as commonly known in the art. In some embodiments, the cushions have an inner covering that is heat sealed to prevent any fluids from entering the foam. Such fluids could be present during the surgical process, or during cleaning.

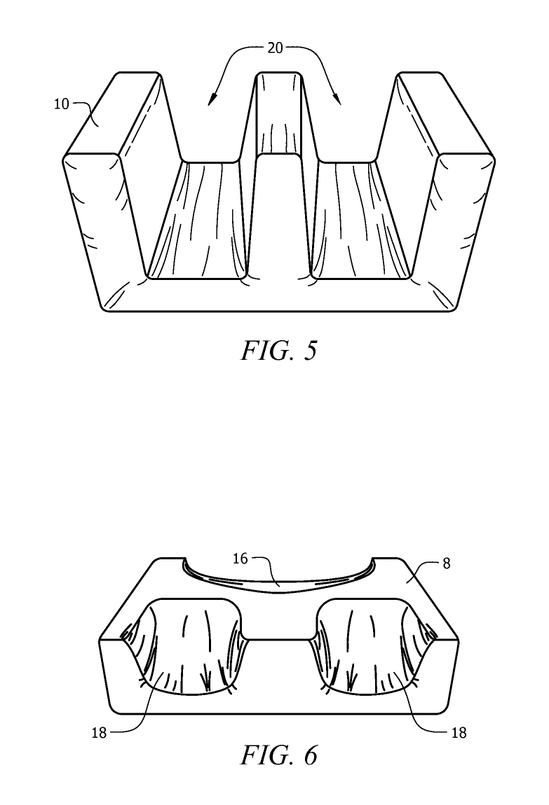

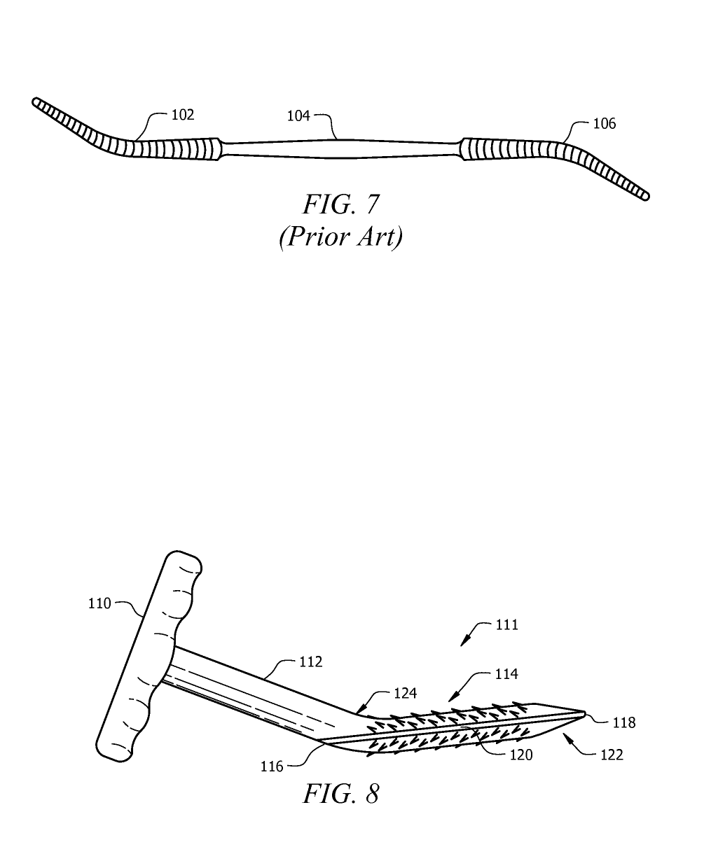

In FIG. 1, the surgeon is prepared to insert the cannulated sacral introducer rasp 112 (see FIGS. 8 and 9) into the spine while guided by the guide wire 130, creating better placement within the spine and fewer steps in surgery.

There exist many different means of removably affixing the cushions to each other, but in this example of the cushion support system, the cushions are held removably affixed to each other by hook-and-loop fasteners. Any means of temporarily affixing the cushions together will constitute removably affixing. Other methods of removably affixing cushions to each other, or any other surface, are anticipated, including hook-and-loop material, snaps, magnets, hooks, and all others as commonly known in the art. Although removably affixing is preferred, in some embodiments some or all of the cushions are permanently affixed to each other. In this example, the main support cushion 6 has a line of hook-and-loop fasteners 40 on one side. The hook-and-loop fasteners on main support cushion 6 interfaces with corresponding hook-and-loop fasteners on the bottom (not shown) of cushions 2 and 8. Also, in this example, the chest height adjustment cushion 4 has hook and loop fasteners on the top 42, and bottom (not shown) to connect to the cushions 2/6 above and below.

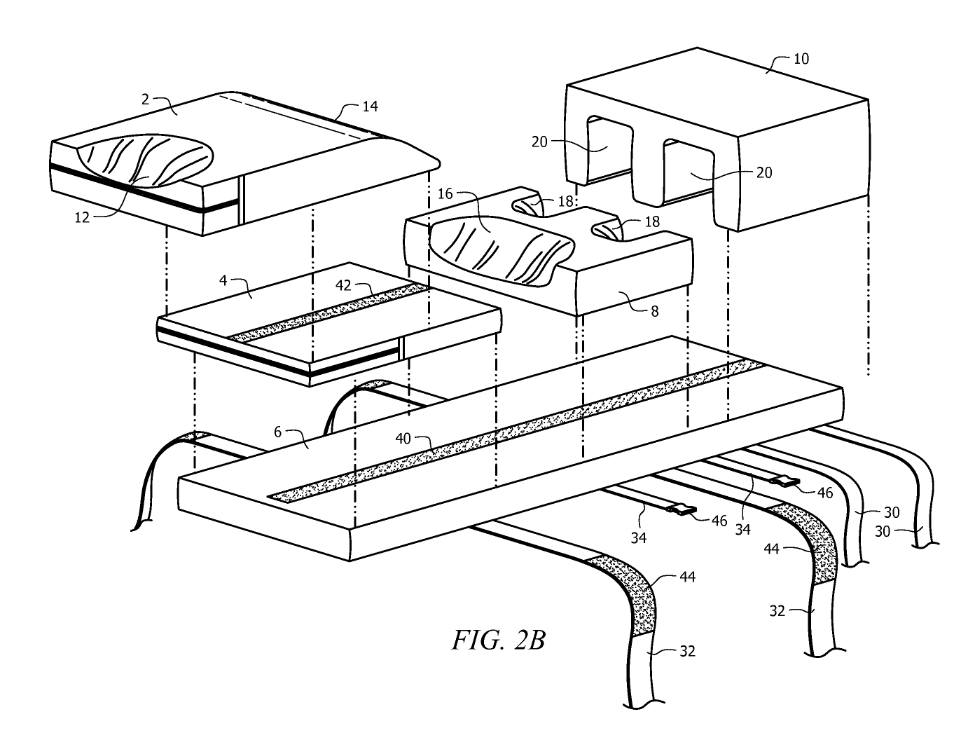

Referring to FIGS. 3A through 3D, the exemplary cushion support system will be further described. FIG. 3A is a side view of the main support cushion 6 and chest support cushion 2. FIG. 3B shows the addition of the chest height adjustment cushion 4 to raise the chest support cushion 2. FIG. 3C shows the addition of the pelvic cushion 8. FIG. 3D shows the addition of the leg isolation and tool support cushion 10, that is held by straps 30 in this example. Other methods of holding the tool support cushion 10 are anticipated, including hook-and-loop material, snaps, magnets, hooks, and all others as commonly known in the art.

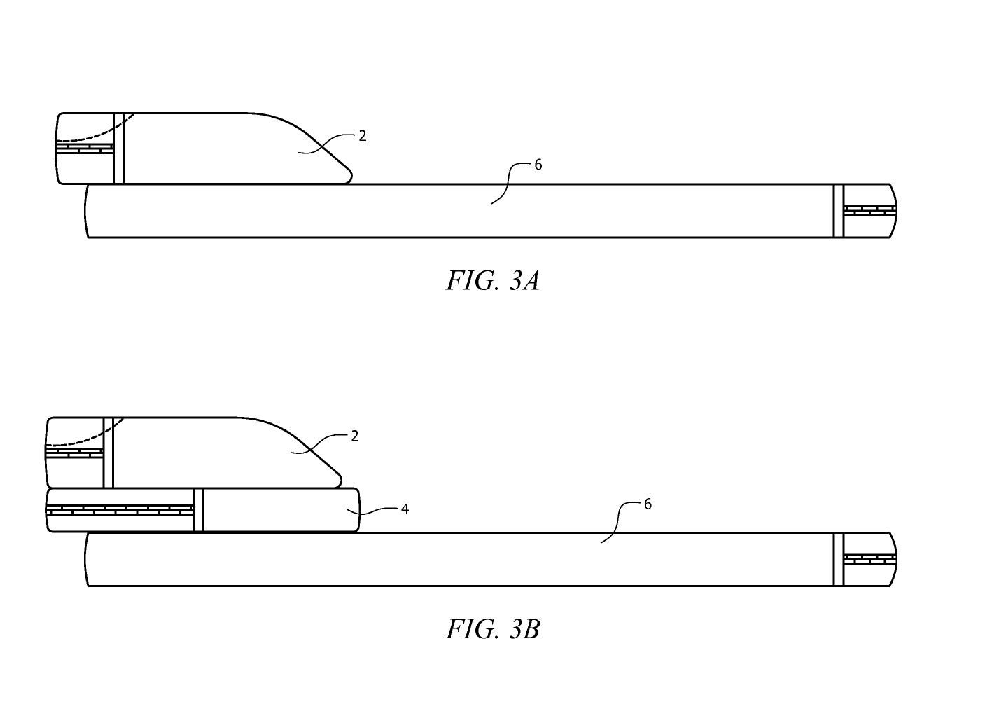

Referring to FIGS. 4A through 4D, the flexibility of the exemplary cushion support system will be described. FIG. 4A shows the cushions 2/4/6/8/10 positioned for an average sized patient. FIG. 4B shows the cushions 2/4/6/8/10 with closer positioning for a shorter patient. FIG. 4C shows the cushions 2/4/6/8/10 positioned for a taller patient.

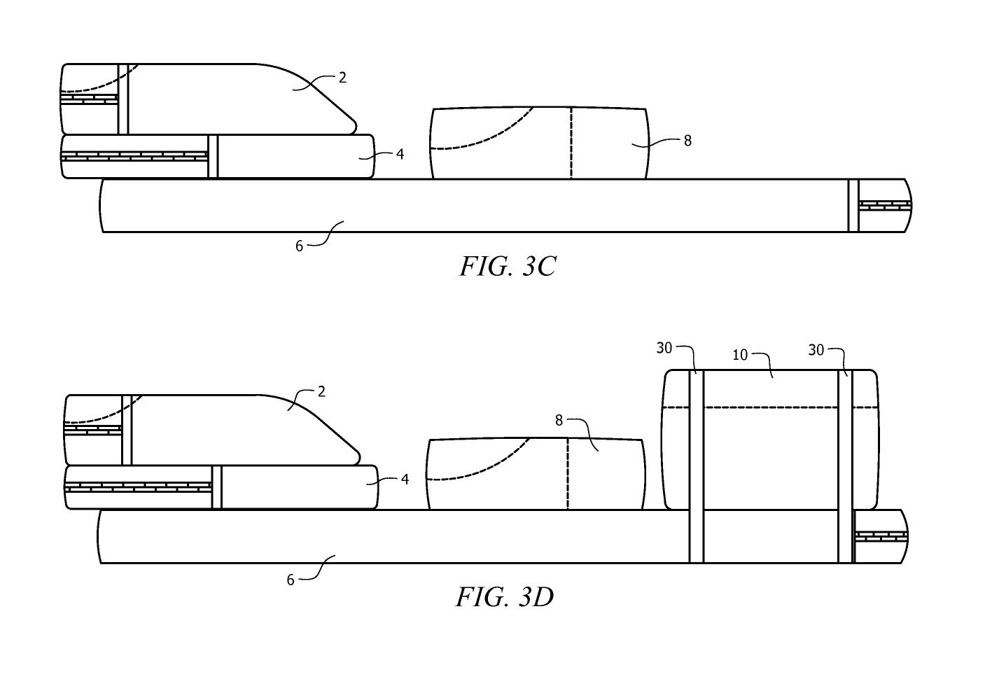

Referring to FIG. 5, the leg isolation and tool support cushion 10 will be described. FIG. 5 shows the bottom of cushion 10 with two passageways 20 for the patient's legs in an inverted position. When the cushion is upright, the large flat surface (visible in FIGS. 1, 2A and 2B) provides a working area for the physician, both for placing instruments that are needed during the procedure and creating a steady platform for hands and arms.

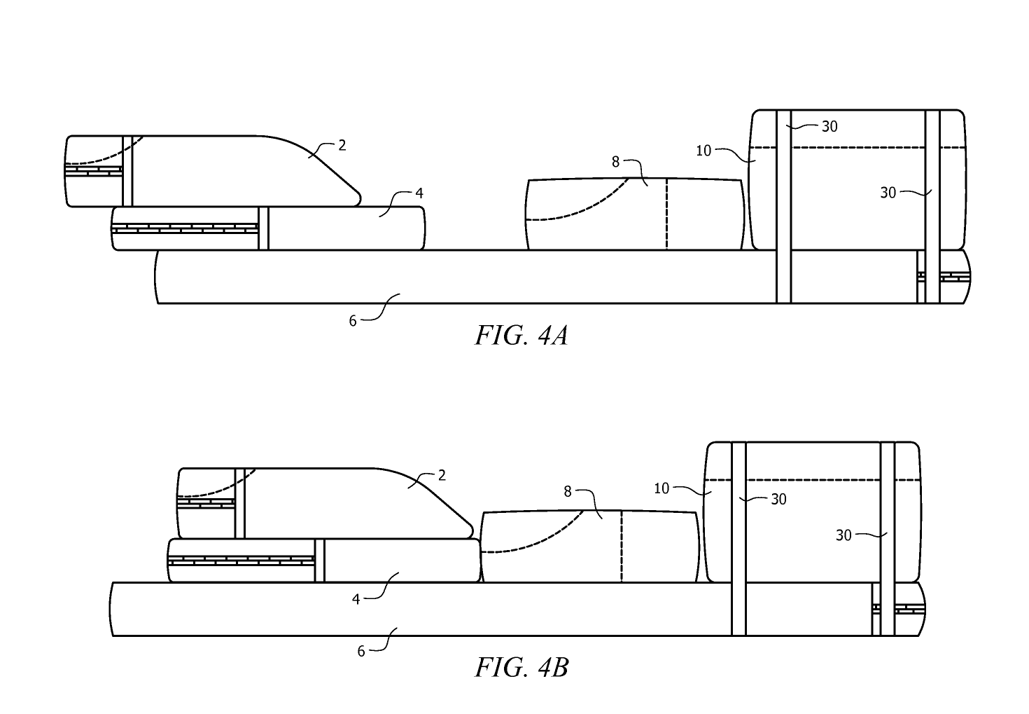

Referring to FIG. 6, the pelvic cushion 8 will be described. The top of the cushion has a first abdominal depression 16 and the two leg depressions 18 for the patient's thighs. The cushion is shaped to support the pelvic bones, with the depressions 16/18 in locations to minimize pressure on the patient's thighs and provide a location for the patient's abdomen to hang.

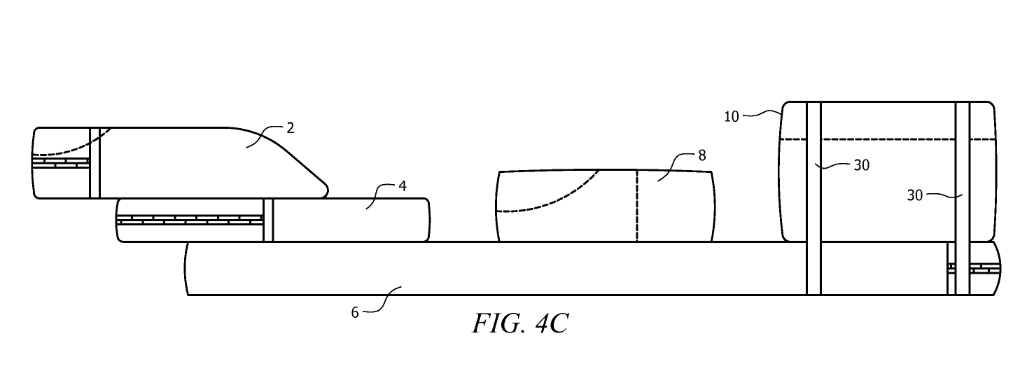

Referring to FIG. 7, the prior art rasp is shown. In the prior art, many rasps are available. The example shown has two curved end sections 102 and 106 with teeth or ridges to remove material when rubbed along the surface. The ends are connected with a smooth section 104 where the rasp is typically held by the physician.

FIG. 8 shows the improved rasp 111. In this example, the cannulated sacral introducer rasp 111 consists of a T-shaped handle 110, a smooth section 112 followed by a barbed section 114, although other handle shapes and configurations are anticipated as known in the art. There is a bore or channel 120 passing preferably, though not required, through the axis of the barbed section. An entrance hole 118 and an exit hole 116 provide access to the bore 120. The entrance hole 118 is preferably near the front tip area 122 (the tip that is distal from the handle area) and the exit hole 116 is preferably near the rear shank area 124. The bore or channel 120 is preferably formed or drilled substantially through the center of the barbed section 114, starting near the front tip area 122 and ending near the rear shank area 124. Other hole 116/118 and bore/channel 120 locations and orientations are anticipated, including channels that are not situated through the center, channels that are not completely enclosed (e.g., a trough on one side of the barbed section), different hole locations, including locations at different sites on the handle 110, shank 112 or barbed area 114 are anticipated. In one embodiment, the holes 116 and 118 are conical openings to facilitate acceptance of a guide wire 130. In some embodiments, the holes 116/118 are of other shapes, including, but not limited to, beveled edges, rounded edges, straight edges, and any other type edge.

The holes 116/118 and bore 120 are preferably sized slightly larger than the guide wire 130, thereby allowing smooth movement of the guide wire 130 through the bore 120. Typically, the guide wire 130 has a circular cross-section and, therefore, the preferred bore 120 also has a circular cross-section, although any bore 120 cross sectional geometry is anticipated to match the cross-sectional geometry of the guide wire 130 such as oval, etc.

In this example of the cannulated sacral introducer rasp 111, the conical end section 122 is smooth. The middle section 114 is covered with an abrasive surface made of smaller triangular barbs. The portion between the middle section 114 and the rear shank area 124 (end section) is covered with another abrasive surface that has larger triangular shaped barbs. Other arrangements of abrasive surfaces are anticipated, including a barbed tip, barbs with shapes other than triangular, barbs along the rear shank area, and any other type of barbs or arrangement of barbs as commonly known in the art.

Referring to FIGS. 9A and 9B, an exemplary use of the cannulated sacral introducer rasp 111 will be described. In order to gain access to the spinal canal, an epidural Tuohey needle (not shown) is inserted into the spinal canal at the sacral hiatus 132. Following the insertion, dye is injected. Next, a guide wire 130 is passed through the needle. In the prior art, the guide wire is then removed and the prior art rasp 104/106, as shown in FIG. 7, is inserted blindly to enlarge the opening in the sacral hiatus 132. The prior art rasp 104/106 is then used to widen the hole created by the needle. In the prior art, because the guide wire must be removed prior to inserting the rasp, the physician is unable to verify the rasp is properly inserted. Additionally, in the prior art, the guide wire is later reinserted after the hole is widened, creating additional steps and increasing the risk of infection.

The cannulated sacral introducer rasp 111 has a channel 120 that runs through the rasp 111, allowing the rasp 111 to slide over the guide wire 130. The rasp is positioned at the entry to the sacrum 132, at the lower end of the lumbar vertebrae 134, without removal of the guide wire 130. The guide wire 130 remains in place during enlargement of the entry channel and, therefore, there is no need to remove the guide wire 130 and reinsert the guide wire 130 later. The rasp 111 is used, for example, to remove ligaments at the base of the spine for spinal penetration by instruments. The hole created by the rasp 111 also gives fluids an easy exit from the spine.

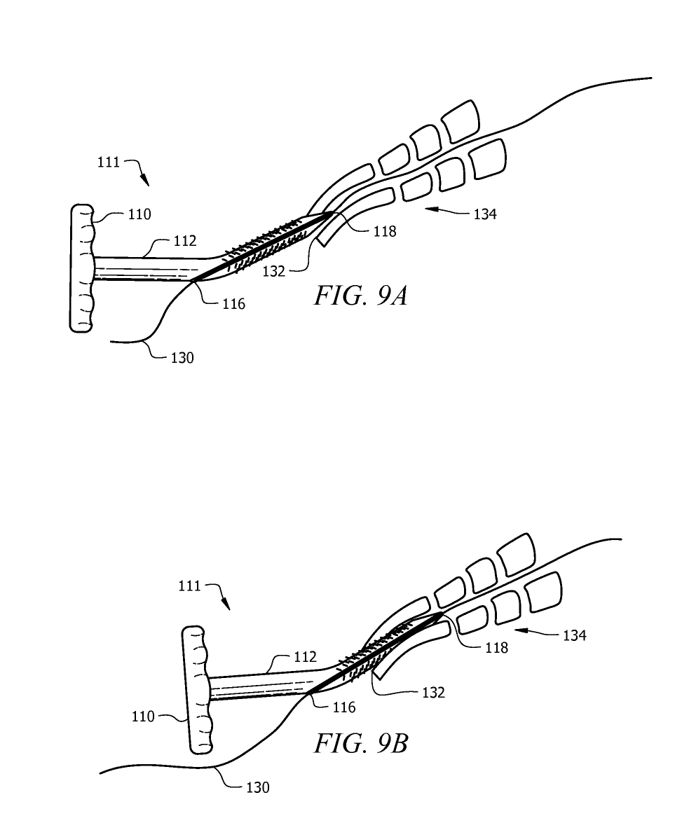

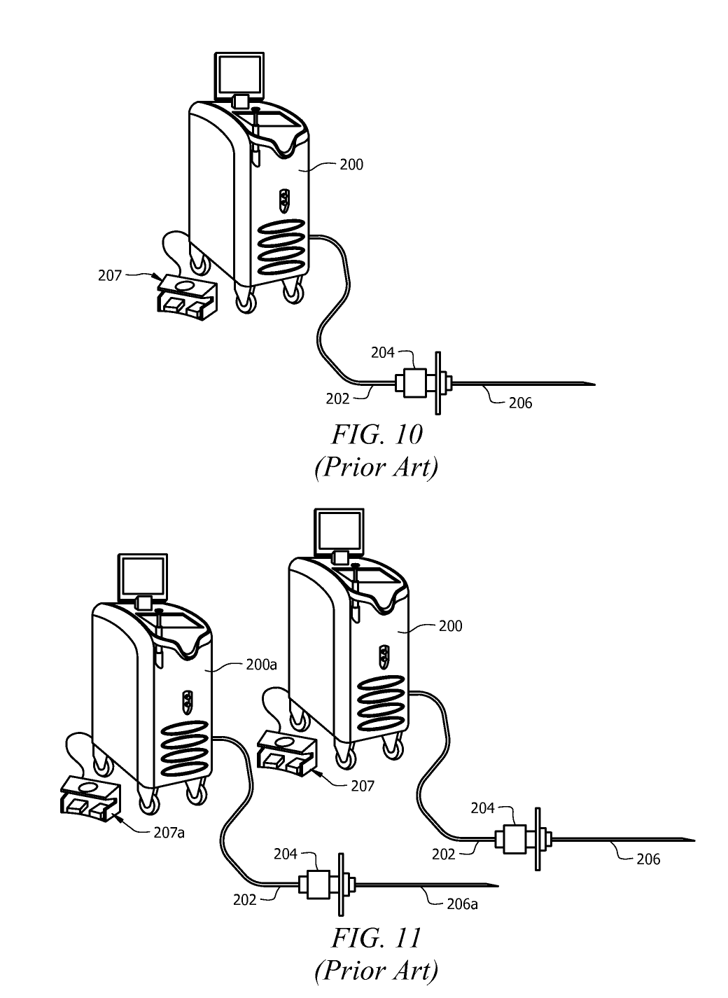

Referring to FIGS. 10 and 11, prior art surgical lasers are shown. Lasers have been used in the past for various types of surgery, including lower back surgery. Laser systems for such procedures are typified by the laser system shown in FIG. 10 consisting of a base system 200 that encloses the electronics used to produce a single wavelength laser beam, a cable 202 containing a fiber optic delivery bundle 206 having one or more individual fiber optic threads and a handle 204. The fiber optic delivery bundle 206 extends beyond the handle for insertion into the patient. The fiber optic delivery bundle directs a single wavelength laser beam at the target tissue within the patient. It is known in the art that different tissues react differently to exposure of high-intensity light radiation of different wavelengths. For example, in “Epiduroscopy” by G. Schültze, methods of performing spinal endoscopy using lasers are described. In this, G. Schültze describes methods for entering the epidural space, guiding a fiber optic probe into the epidural space with the help of a C-arm device and correcting various situations using the laser. In chapter 7.5, G. Schültze discusses the Epidural laser adhesiolysis, for example, using a 1064-nm Nd laser, a YAG 1320-nm Nd laser, and a 940-nm laser for “coagulation of bleeding, rechanneling stenosis caused by tumors and destroying plaques in vessel walls.” In this procedure, a fiber optic cable is introduced into the epidural space via a working channel of an epiduroscope under epiduroscope vision. A laser diode of from 1 watt to 25 watts fires a burst of energy of the specific wavelength through the fiber and onto the target tissue. G. Schültze describes that the light energy penetrates the tissues but is not significantly absorbed by the surrounding hemoglobin, melanin or water (see FIG. 17). The prior art laser 200 of FIG. 10, having a single wavelength output is very useful in removing a single type of tissue.

In these procedures, when multiple wavelengths of laser are needed to remove different types of tissue (e.g. hydrated bulging disc tissue as opposed to desiccated, degenerated disc tissue), multiple laser systems 200 of the prior art were used as shown in FIG. 11. During the prior art procedures, the fiber optic bundle 206 from the first laser system 200 is inserted into the epidural area of the patient and the laser system 200 is triggered through a foot switch 207 to radiate the first type of tissue (e.g. ligament tissue, or well hydrated bulging disc tissue) with a prescribed power of the first wavelength of light, thereby vaporizing that first type of tissue. Now, if a second type of tissue is encountered (e.g. dessicated, degenerated disc tissue), the first fiber optic bundle 206 is pulled out from the epidural area and a second fiber optic bundle 206A is inserted. The second fiber optic bundle 206A is interfaced to a second laser system 200A (as shown in FIG. 11) which emits a different wavelength of laser light than that of the first laser system 200. Again, the laser system 200A is triggered through a second, distinct foot switch 207A to radiate the second type of tissue (e.g. dessicated, degenerated disc tissue) with a prescribed power of the second wavelength of light, thereby vaporizing that second type of tissue. During a single operation, it is often required to repeat these steps as different types of tissue are exposed and need to be removed.

In addition to requiring extra steps of removal and insertion by the surgeon into and out of the patient, increasing the opportunity for infection, having two or more laser systems 200/200A increases cost because many of the components of the first laser system 200 are duplicated in the second and subsequent laser systems 200A.

Additionally, using sequentially inserted fiber optic delivery bundles 206 do not allow maintaining patency of the epidural space by filling the epidural space with fluid being that the patency is lost each time the first fiber optic delivery bundles 20 is removed and replaced with the second fiber optic bundle 206A.

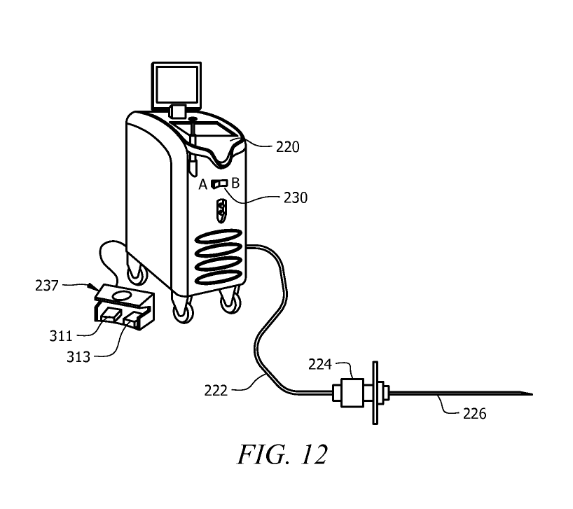

Referring to FIG. 12, the multiple wavelength surgical laser 220 is shown. The multiple wavelength surgical system 220, shown in an exemplary enclosure, emits two or more different wavelengths of light (e.g. laser light) radiation through a single cable 222 having one or more fiber optics within a fiber optic bundle 226 that deliver the laser energies to the abnormal tissue within the patient. With this, the epidural space is filled with a fluid such as, but not limited to saline, to maintain patency. Although many fluids are anticipated, saline is preferred because of the similarity of saline to composition of other cells of the body.

Filling the epidural space with a fluid (e.g. saline) provides several advantages during laser assisted procedures using the multiple wavelength surgical laser 220. One advantage is that the fluid maintains patency of the epidural space, thereby improving visibility and maneuverability.

Another advantage is that the fluid absorbs excess laser energy which is important, for example, when the target vaporizes or the surgeon misses the target. In such, the laser energy is dissipated so as to not significantly damage tissue behind the target or further down the epidural space. For example, if the target is vaporized, yet the laser energy continues, the laser energy attenuates to a relatively safe level in as little as 0.3 mm from the tip of the fiber optic bundle 226. Another advantage is improved accuracy of addressing the target tissue with the laser energy through improved visibility and space for the fiber optic bundle 226 to travel.

Since the epidural space is now filled (or substantially filled) with a fluid, the wavelengths of laser energy emitted by the light emitting device 310/312 must not significantly react with the fluid. For example, when the fluid is saline, wavelengths of laser energy that resonate with saline need be avoided because firing of that light emitting device 310/312 will emit sufficient light energy at that resonate frequency (or range of frequencies) to cause the saline to rapidly vaporize. Such rapid vaporization in such a confined space (epidural space) creates a shock wave through the spinal column, potentially damaging the spinal cord, brain stem, retina, eyes, brain, etc.

Although shown having a specific handle 224 and cable system 222/226, any construction of the two or more wavelengths of laser radiation is anticipated.

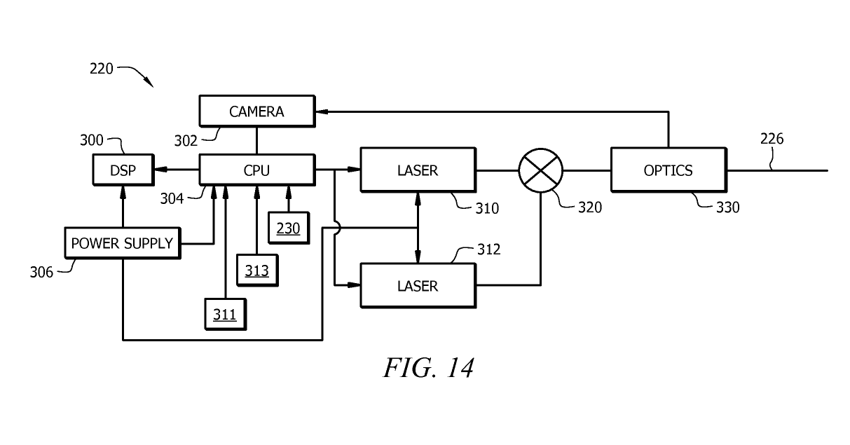

A light emitting device 310/312 (see FIG. 14) is any device that provides light, the light being able to be focused into a beam and the light sufficient (e.g. high power) to affect targeted tissue. An example of such a device is a laser 310/312, but there is no limitation that lasers are the only allowable source of energy or light energy. Any light emitting device can be substituted, or devices that provide other sources of focused energy, including energy not classified as light.

In order to be useful, the light emitting device needs to emit sufficient energy as to affect the targeted tissue, referred to in this description as “high-power.” Using a laser as an example, existing laser light emitting devices have power outputs that range from, for example, a 1 mW laser pointer to a 100 kW or greater laser used in weaponry and research applications. To be effective for surgical use, a laser 310/312 (or other light output device) needs to produce sufficient power output as to affect the target tissue while not damaging surrounding tissue or other parts of the patient's body. Light power outputs in the range of 1-25 watts have been shown useful in affecting many types of unwanted mammalian tissue. The interaction between the light source and tissue will vary depending upon the type of light utilized, the wavelength, the duration of light emission, the light generator source, the power level, pulsed vs. non-pulsed deliver of the light, and the energy field created (i.e., direct surface contact with light, or heating of surrounding tissue and possible formation of a steam bubble with subsequent tissue vaporization). Again, the fluid introduced into the epidural space reduces risks associated with using a high-energy source within the epidural space.

Many existing surgical laser systems 200/200A provide for controls to adjust the output power of the laser. It is anticipated that, in some embodiments, the multiple wavelength surgical laser 220 also has an adjustment to control the power output of each individual source 310/312 of laser radiation, or to simultaneously control the individual sources 310/312 by application of a common ratio of power between all sources 310/312. All types of on/off and power setting controls are anticipated, including foot switches, voice control, a pressure switch, eye recognition, computer control, etc. In the example shown, a single foot switch module 237 has separate activation switches 311/313 for each laser (two are shown 311/313, though any number are anticipated to match the number of sources 310/312).

Instead of alternating between fiber optic bundles 206/206A of the prior art, a single fiber optic bundle 226 delivers multiple wavelengths of laser radiation to the target tissue. This allows for filling of the epidural space with a fluid such as saline to maintain patency and dampen excess energy from the sources 310/312, etc. Therefore, it is important that, once the epidural space is filled with fluid and the fiber bundle 226 is inserted into the epidural space, there is no need to remove the fiber bundle 226 when a different wavelength of light is needed. Thus, the need for multiple wavelengths of light through a single fiber bundle 226. As stated above, it is also important to select the wavelengths of light to limit interaction and vaporization of the fluid.

The wavelength of laser radiation passing through the fiber optic bundle 226 is controlled by switching from one laser source 310/312 to a different laser source 310/312 by, for example, a selector switch 230 or different foot switches 311/313 within a foot pedal 237 or any other mechanism known in the art. In some embodiments the wavelengths are delivered simultaneously at a fixed or variable ratio of power, as desired and set by the laser operator.

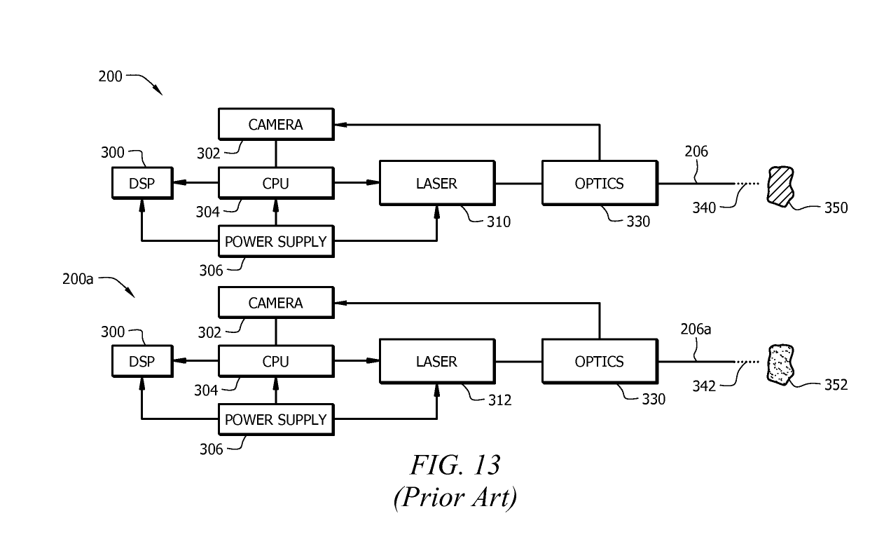

Referring to FIG. 13, a block diagram of a prior art surgical laser is shown. In the prior art, two different laser systems 200 were employed, each duplicating most or all of the components of the other and each delivering their wavelength of laser radiation through one or more fiber optics within a fiber bundle 206. Each of the prior art laser systems 200/200A had its own power supply 306, display 300, processor 304, optics 330 and light (laser) radiation generator 310/312 such as a laser diode 310/312 or any other source of laser radiation. In the example of the prior art shown, the first laser systems 200 has a first laser 310 emitting a first wavelength of laser radiation that is best for use with vaporizing a first type of tissue 350 (e.g. ligament, or well hydrated bulging disc tissue). The second laser systems 200A has a second laser emitting a second wavelength of laser radiation that is best for use with vaporizing a second type of tissue 352 (e.g. dessicated, degenerated disc tissue).

As stated before, the systems of the prior art required the surgeon to pull out one laser fiber bundle 206 and insert another laser fiber bundle 206A when operating on a different type of tissue. This makes it difficult to keep the epdirual space filled with a fluid to maintain patency, etc. FIG. 13 shows the duplication of components found in the prior art such as the display 300, the camera 302, the processor 304, the power supply 306, the optics 330 and the fiber bundle 206.

Referring to FIG. 14, a block diagram for the new multiple wavelength surgical system 220 is shown. The multiple wavelength surgical systems 220 has a power supply 306, a display 300, a processor 304, optics 330 and two or more light (e.g. laser) radiation generators 310/312 such as a laser diode 310/312 or any other source of directed light radiation. Each light radiation generator 310/312 delivers their respective wavelength of light radiation through one or more fiber optics within a single fiber bundle 226. In use, the epidural space is opened, in some embodiments using a rasp, the epidural space is filled with a fluid (e.g. saline), and then the fiber optics are guided into the epidural space, preferably with the help of a C-arm device. One or multiple sources of light 310/312 are fired as needed to correct various situations such as to remove a first type of tissue (e.g. ligament tissue, or well hydrated bulging disc tissue) with a prescribed power of the first wavelength of light, thereby vaporizing that first type of tissue or to remove a second type of tissue (e.g. dessicated, degenerated disc tissue) with a prescribed power of the second wavelength of light, thereby vaporizing that first type of tissue, etc. In the example shown, laser radiation from each of the laser radiation generator 310/312 are mixed or switched into the fiber optic bundle 226 by a light mixer 320 or light switch 320 as known in the industry. Alternately, the laser radiation from each of the laser radiation generator 310/312 is interfaced to its own, dedicated fiber optic within the single fiber optic bundle 226. Any number of laser radiation generators 310/312 is anticipated. Multiple wavelengths can be delivered independently or simultaneously through the fiber optic. Again, the system is not limited to any particular source of light and lasers 310/312 are examples of such sources of light.

The first laser radiation source 310 emits a first wavelength of laser radiation that is best for use with vaporizing a first type of tissue 350 (e.g. ligament or scar tissue), for example approximately 980 nanometers. The second laser radiation source 312 emits a second wavelength of laser radiation that is best for use with vaporizing a second type of tissue 352 (e.g. disc tissue), for example approximately 1480 nanometers. In embodiments with three wavelengths, a third laser radiation source (not shown) emits a third wavelength of laser radiation that is best for use with vaporizing a third type of tissue (not shown), for example approximately 2100 nanometers, and so forth. Again, any number of laser radiation sources 310/312 is anticipated. Any number of wavelengths can be delivered independently or simultaneously through the fiber optic.

In some embodiments, the laser radiation from the two or more laser sources 310/312 is either combined or switched by a light mixer/switch/multiplexor 320 and directed into one or more fiber optic fibers 226 through an optical system 330 as known in the industry. In other embodiments, laser radiation from each of the two or more laser sources 310/312 is directed into its own set of one or more fiber optic fibers 226 through an optical system 330 as known in the industry.

To control which of the laser radiation generator 310/312 is selected and subsequently excited to deliver its respective wavelength of laser radiation, a control 230/318 is provided such as a selector switch 230 or multiple floor switches 311/313, etc. For example, when the surgeon needs the first wavelength of laser radiation, the surgeon moves the selector switch 230 to a first position then initiates emission of the laser energy by, for example, pressing the foot switch 311/313 with a foot. All types of control are anticipated, including foot switches, voice control, pressure switches, eye recognition, computer control, etc. When the surgeon needs the second wavelength of laser radiation, the surgeon moves the selector switch 230 to a second position, then initiates emission of the laser energy by, for example, pressing the foot switch 311/313 with a foot. In another embodiment, when the surgeon needs the first wavelength of laser radiation, the surgeon initiates emission of the laser energy by pressing a first switch 311 the foot switch 237 with a foot. When the surgeon needs the second wavelength of laser radiation, the surgeon initiates emission of the laser energy by, for example, pressing a second switch 313 of the foot switch 237 with a foot. Many ways are known to control the emission of the laser energy, all of which are included here within. In embodiments in which multiple wavelengths of laser energy are concurrently delivered, one or more switches (not shown for brevity purposes) or foot switches (not shown for brevity purposes) are provided for concurrently delivering two or more of the wavelengths of laser energy at the same time. The individual sources 310/312 are individually or simultaneously controlled by application of a common ratio of power between the sources 310/312.

Referring to FIG. 15, a graph 300 of absorption by materials of ranges of wavelengths of light is shown. This graph illustrates absorption of two different materials (M1 and M2). As shown by the graph 300, a first material containing water M1 (e.g. H₂O) readily absorbs laser energies above around 800 nano meters, but poorly absorbs laser energies between around 200 nano meters and 800 nano meters. A second material containing Hemoglobin M2 (e.g. H₂O₂) readily absorbs laser energies between 200 and 600 nano meters, but poorly absorbs laser energies above 600 nano meters. Therefore, laser energy from a 532 nano meter laser W1 will be highly absorbed by tissue containing Hemoglobin but will barely be absorbed by tissue containing water, while laser energy from a 982 nano meter laser W2 will be absorbed by tissue containing either water or hemoglobin, and laser energy from a 2100 nano meter laser W3 will be absorbed by tissue containing water but poorly absorbed by tissue containing hemoglobin. This is why different lasers are useful for vaporizing different classes of tissue.

While the application addresses the system, method, and device in terms of the use of lasers to produce light, there is no limitation that lasers are the only allowable source of energy or light energy. Any light emitting device can be substituted, or other sources of focused energy, including energy not classified as light, may be used in the same manner.

Additionally, the application addresses specific frequencies as exemplary due to the commercial availability of certain laser light frequencies. It is anticipated that as lasers become commercially available in other frequencies of light that they will be used within the system, method, and device to accomplish tissue removal.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for performing surgery within the epidural space of a patient, the epidural space filled with a fluid before performing the surgery, the system comprising:
a plurality of high-power light emitting devices, each of the high-power light emitting devices emitting light energy of a different wavelength selected from the group consisting of 980 nanometers, 1480 nanometers and 2100 nanometers;
a means for controlling the high-power light emitting devices, initiating output of the light energy under user control; and
one or more fiber optics, a first end of the fiber optics accepting the light energy from the plurality of high-power light emitting devices and a distal second end delivering the light energy to target tissue within the epidural space of the patient;
wherein a first type of tissue is vaporized using the light energy from a first high power light emitting device of the plurality of high-power light emitting devices and a second type of tissue is vaporized using the light energy from a second high power light emitting device of the plurality of high-power light emitting devices without the need to remove the fiber optics from the epidural space, thereby maintaining the fluid within the epidural space.

2. The system for performing surgery of claim 1, wherein the high-power light emitting devices are a plurality of lasers, each laser of the plurality of lasers emitting a different wavelength of light.

3. The system for performing surgery of claim 1, wherein the means for controlling is a foot switch having a control for each of the high-power light emitting devices, each control operatively coupled to a different one of the high-power light emitting devices such that operation of one of the controls initiates output of the light energy from a corresponding high-power light emitting device.

4. The system for performing surgery of claim 3, wherein operation of multiple controls initiates output of the light energy concurrently from multiple high-power light emitting devices.

5. The system for performing surgery of claim 1, wherein the fluid is saline.

6. The system for performing surgery of claim 2, wherein the lasers are solid-state laser diodes.

7. The system for performing surgery of claim 1, wherein the one or more fiber optics is one fiber optic and the light energies from each of the plurality of high-power light emitting devices are mixed into the one fiber optic.

8. The system for performing surgery of claim 1, wherein the light energy from each of the plurality of high-power light emitting devices is directly connected to one or more dedicated fibers within the fiber optics.

9. The system for performing surgery of claim 1, wherein the plurality of high-power light emitting devices is three high-power light emitting devices, a first high-power light emitting device of the three high-power light emitting devices emitting light energy of 980 nanometers, a second high-power light emitting device of the three high-power light emitting devices emitting light energy of 1480 nanometers, and a third high-power light emitting device of the three high-power light emitting devices emitting light energy of 2100 nanometers.

10. A method of for performing surgery within the epidural space of a patient comprising:
opening a port to access the epidural space;
filling the epidural space with a fluid;
providing a high-power light source, the high-power light source comprising:
a plurality of high-power light emitting devices, each of the high-power light emitting devices emitting light energy of a different wavelength than the other high-power light emitting devices, wherein the wavelengths are selected from the group consisting of 980 nanometers, 1480 nanometers and 2100 nanometers;
a plurality of switches, each of the switches controlling a different one of the high-power light emitting devices, initiating output of the light energy by user control; and
a bundle of one or more fiber optics, a first end of bundle accepting light energy from the high-power light emitting devices and a distal second end of the bundle delivering the light energy;
inserting the distal second end of the bundle into the epidural space of the patient;
aiming the distal second end of the one or more fiber optics at a first type of tissue;
activating a first switch of the switches, thereby emitting light from a first high-power light emitting device of the high-power light emitting devices to affect the first type of tissue;
without removing of the bundle, aiming the distal second end of the one or more fiber optics at a second type of tissue; and
activating a second switch of the switches, thereby emitting light from a second high-power light emitting device of the high-power light emitting devices to affect the second type of tissue;
whereas the fluid attenuates excess energy from the plurality of high-powered light emitting devices.

11. The method of claim 10, wherein the high-power light emitting devices are a plurality of lasers, each laser of the plurality of lasers emitting a different wavelength of light.

12. The method of claim 11, wherein the lasers are solid-state laser diodes.

13. The method of claim 10, wherein the plurality of high-power light emitting devices is three high-power light emitting devices, a first high-power light emitting device of the three high-power light emitting devices emitting light energy of 980 nanometers, a second high-power light emitting device of the three high-power light emitting devices emitting light energy of 1480 nanometers, and a third high-power light emitting device of the three high-power light emitting devices emitting light energy of 2100 nanometers.

14. The method of claim 10, wherein the fluid is saline.

15. A system for performing surgery within the epidural space of a patient, the system comprising:
a means for opening an access to the epidural space;
a means for substantially filling the epidural space with a fluid;
a first source of laser light energy, the first source of laser light energy emitting light at 980 nanometers;
a first switch coupled to the first source of laser light energy, the first switch initiating light output from the first source of laser light energy when operated;
a second source of laser light energy, the second source of laser light energy emitting light at 1480 nanometers;
a second switch coupled to the second source of laser light energy, the second switch initiating light output from the second source of laser light energy when operated; and
a bundle of one or more fiber optics, a first end of the bundle accepting light energy from the first source of laser light energy and from the second source of laser light energy and a distal second end of the bundle delivering the light energy to target tissue within the epidural space of the patient;
wherein a first type of tissue is vaporized using the light energy from the first source of laser light energy and a second type of tissue is vaporized using the light energy from the second source of laser light energy without the need to remove the fiber optics from the epidural space.

16. The system for performing surgery of claim 15, wherein concurrent operation of both the first switch and the second initiates simultaneous output of the light energy of both the first source of laser light energy and the second source of laser light energy.

17. The system for performing surgery of claim 15, further comprising a third switch that initiates simultaneous output of the light energy of both the first source of laser light energy and the second source of laser light energy.

18. The system for performing surgery of claim 15, further comprising a third source of laser light energy and a third switch coupled to the third source of laser light energy, the third switch initiating light output from the third source of laser light energy when operated, the third source of laser light energy emitting light at 2100 nanometers.

19. The system for performing surgery of claim 15, wherein the fluid is saline.

20. The system for performing surgery of claim 19, wherein the saline attenuates at least some of the light energy within the epidural space.
